(19) United States
(12) Patent Application Publication
(10) Pub. No.: US 2011/0275960 A1
(43) Pub. Date: Nov. 10, 2011

(54) ENTRANCE INFORMATION SYSTEM AND METHOD FOR ISSUING ENTRANCE INSTRUCTIONS FOR A SLEEPING ROOM BY AN ENTRANCE INFORMATION SYSTEM

(75) Inventors: Joanne Henriette Desiree Monique Westerink, Eindhoven (NL); Felix Henric Govert Ogg, Eindhoven (NL); Ronaldus Maria Aarts, Eindhoven (NL)

(73) Assignee: KONINKLIJKE PHILIPS ELECTRONICS N.V., EINDHOVEN (NL)

(21) Appl. No.: 13/144,496

(22) PCT Filed: Jan. 22, 2010
(86) PCT No.: PCT/IB10/50282

§ 371 Date: Jul. 14, 2011

(30) Foreign Application Priority Data
Jan. 28, 2009 (EP) 09151530.4

(51) Int. Cl. A61B 5/00

(52) U.S. Cl. 600/595

(57) ABSTRACT

The present invention relates to an entrance information system and a method for operating an entrance information system issuing an entrance instruction for a sleeping room in dependence of a sleeping signal from a sleep detector detecting the sleep stage of a person inside the sleeping room.

The present invention relates to an entrance information system and a method for operating an entrance information system issuing an entrance instruction for a sleeping room in dependence of a sleeping signal from a sleep detector detecting the sleep stage of a person inside the sleeping room.

It has become common knowledge that the sleep of human beings iterates between three phases: a REM (Rapid Eye Movement) phase, a deep sleep phase and a shallow sleep phase. The Rapid Eye Movement (REM) phase is high quality sleep and is usually linked to our dreaming episodes. Both the REM sleep and the deep sleep are high quality sleep and one is not easily awakened during these sleeping phases. But if one does get woken up, one usually feels uncomfortable afterwards, and has missed the valuable restoration that sleep brings. On the contrary, shallow sleep is only a light sleep during which it is easy to get awoken by outside signals. If it is necessary to wake someone up, it is best to do it during this period. The shallow sleep period usually lasts longer that any of the other two periods. A method and an apparatus in which an REM sleep phase is accurately identified by detecting and counting s selectable number of eye movements within a selectable time interval is known from the prior art document U.S. Pat. No. 4,863,259 A, for example.

Furthermore, a prior art solution to wake up a person in dependence of the current sleeping phase is disclosed by the document WO 2008/0 989 423 A2, wherein an intelligent alarm clock arrangement is adapted to an electric apparatus adjacent to the head of a sleeping person for detecting light sleep, arousal or the wake state of the sleeping person and to perform an awakening alarm during a pre-programmed time window, if there are arousals, awake state and movements present at that time to induce the awakening at a biologically advantageous time.

Generally, human beings should have sleep of sufficient quality to feel well during daytime. This also holds for persons who are ill and for little children, maybe even more so. However, these persons also often require the careful attention of others, (e.g. hospital staff, parents), who will have to enter the sleep area for this reason. This will enhance the risk that they will inadvertently wake the sleeping person. This is probably not beneficial for their health and well-being. These same caregivers will need to wake the sleepers at other occasions. Again it would serve the best interest of the sleeping person if they could do so at a moment that is minimally inconvenient, e.g. preferably not during REM or deep sleep phases. Without any further tools, however, it is very difficult for the caregivers to know whether now is an optimal timing to awake someone in a sleeping room, or—in case they want to inspect them without waking them—whether now is a relatively safe time to go into the sleeping room of the sleeping person. Particularly, it is not possible for the caregivers to appraise the current sleeping phase of the sleeping person in a sleeping room, if the caregivers are outside the sleeping room. Furthermore, there is the risk that opening the door by the caregiver will already wake up the sleeping person, if the person is currently in the shallow sleep phase.

It is therefore an object of the present invention to provide an entrance information system and a method for issuing entrance instructions for a sleeping room informing a second person, particularly a caregiver, outside the sleeping room whether the sleeping persons is in a phase of high quality sleep or not.

The above object is accomplished by an entrance information system for a sleeping room comprising a sleep detector for detecting the sleep stage of a person in the sleeping room, wherein the sleep detector is provided inside the sleeping room, and an output unit for issuing an entrance instruction for the sleeping room in dependence of a sleeping signal from the sleep detector, wherein the output unit is provided outside the sleeping room. Beneficially, a person outside the sleeping room will be informed of the current sleeping phase of the sleeping person inside the sleeping room by the entrance instruction of the output unit without opening the door of the sleeping room. Therefore, this person can decide entering the sleeping room immediately or later in time without disturbing the sleeping person. The sleep detector in the sense of the present invention comprises a sensor unit, like a camera, a microphone, an electroencephalogram (EEG), an electrocardiogram (ECG), a pulse monitor or the like. For example, the sleep detector comprises an apparatus for detecting sleep states as disclosed in the above mentioned document U.S. Pat. No. 4,863,259 A. Particularly, the sleep detector is combined with a control unit for receiving and analyzing the sleeping signal from the sleep detector and for distinguishing the current sleep phase from the current sleeping signal. In this case, the control unit further determines the entrance instruction in dependency of the current sleep phase. The entrance instruction is reflected by the output unit which is positioned at a certain distance from the sleeping person. The output unit therefore reflects the sleep state of the sleeping person, so that the caring persons can take the sleep state into account in taking the decision whether to enter the room or not, for example. Also in the case the person outside the sleeping room wants to wake up the sleeping person, an optimal timing for that is advised by the entrance instruction. The entrance instruction can be a visual, an acoustic and/or a haptic signal. Preferably, the output unit features a display unit and/or an illuminated sign close to the door of the sleeping room which illuminates in a certain color in accordance to the current sleep phase. Preferably, the entrance instruction is displayed as a text message, for instance a running text. Alternatively, the display is provided in a control room or in a ward room, for example. Sleeping room in the sense of the present invention comprises every kind of room which allows at least one person to sleep, to relax, to rest, to retire, to recuperate or the like. For instance, the sleeping room does not necessarily needs a bed, a sickbed, a couch or the like.

In a preferred embodiment the display unit outside the door reflects the sleep state and combines it with a warning. For example, in case of REM sleep the display unit notifies: “The sleeper is in REM sleep: Safe to go in quietly, but waking not advised.”. In case of deep sleep the display unit notifies: “The sleeper is in deep sleep: Safe to go in quietly, but waking not advised.”. In case of shallow sleep the display unit notifies: “The sleeper is in shallow sleep: This is a good time to wake the sleeper if you need to; otherwise entering not advised.”. And in case of an awake person: “The person is awake. Entering quietly might be appreciated anyway”.

In a preferred embodiment of the present invention, the entrance information system comprises a user interface for setting up the control unit. Beneficially, the sleeping person and/or the caregivers can input possible preferences of the sleep. Possible preference can be a wakeup time, a time interval for the wakeup time and/or personal information of the sleeping person, like name, age, sex, height, weight, disease and the like. Particularly, certain parameters of the personal information are taken into account by the control unit to distinguish which of the three sleep phases the sleeper is currently in. In a preferred embodiment the display unit outside the door reflects the sleep state and combines it with the preferences of the sleeping person. For example, in case that the sleeper prefers to be left asleep at this hour of day the display unit notifies: “The sleeper is in shallow sleep and prefers to be left sleeping at this hour: If you need to wake the sleeper this is a good time; otherwise entering not advised.”. And in case that the sleeper prefers to be woken up at this hour of day the display unit notifies: “The sleeper is in shallow sleep and prefers to be woken up at this hour: Now is a good time to do so.”.

Preferably, the connection between the sleep detector and the control unit and/or between the control unit and the output unit and/or between the control unit and the user interface is wireless. Beneficially, the subsequent integration of the entrance information system based on wireless connections is comparatively simple and cost-effective.

In a preferred embodiment the entrance information system comprises a plurality of sleep detectors for detecting the sleep stage of a plurality of persons in the sleeping room. In case of multiple sleepers in one room, the message generator could combine their individual preferences and sleep states. In case one of them is in shallow sleep and does not want to be woken up, entering the room is not advised for either of them.

In a preferred embodiment the control unit incorporates a recorder, wherein the time-dependant course of the sleeping signal and/or of the entrance instruction is recorded by the recorder. Beneficially, the care giver can see how the sleep quality of the sleeper has been in a previous period.

Preferably, the entrance instruction system for a sleeping room is used in a hospital, a hotel, a nursing home, a retirement home, a nursery or the like. This invention could be applied in all circumstances where care givers sometimes need to wake sleeping people, or to enter areas in which other people sleep. This includes patient rooms in hospitals, where the staff sometimes need to wake people, and at other times need to enter the room with the intent to let them sleep. The same holds for a home situation in which a parent wants to inspect and/or wake the sleeping children. The sleeping children application can also be very useful in day-care centres, where children regularly take a nap. Furthermore, the present invention can be used in hotels, where it is an extra warning to cleaning personnel that inside the room someone is sleeping. Lastly, one could think of use in airplanes, where sleeping persons can be waked at a convenient moment.

In a preferred embodiment the output unit further comprises a door locking device.

Another object of the present invention is a method for issuing entrance instructions for a sleeping room by an entrance information system, wherein the entrance instruction for the sleeping room are allocated in dependence of the sleeping signal of the sleeping detector and issued by the output unit. Preferably, the entrance instruction permits the quiet entrance in the sleeping room during the REM and the deep sleep phases of the person and/or prohibits the entrance in the sleeping room during shallow sleep phase of the person and/or permits waking up the person sleeping in the sleeping room during shallow sleep phase of the person.

Preferably, an emergency call is activated by the control unit in case of abnormalities in the sleeping signal, e.g. abnormalities in the EEG. The emergency call is provided to warn the caregiver and/or to wake up the sleeping person automatically. Another possibility is to use the display unit and/or the control unit for non-sleep related messages, that might nevertheless be useful to the care taker and care giver alike, e.g. a message ‘please clean room’, to be entered by the sleeper when he is temporarily leaving the room.

In a preferred embodiment the entrance instruction is allocated in dependance of a plurality of sleeping signals from a plurality of sleeping detectors in the sleeping room for detecting the sleep stage of a plurality of persons in the sleeping room. For example, the entrance instruction prohibits the entrance in the sleeping room, if at least one of the plurality of persons in the sleeping room will sleep in the shallow sleep phase, and/or permits the entrance in the sleeping room, if every person in the sleeping room will sleep in the REM or in the deep sleep phase.

In a preferred embodiment the sleep state of each person of the plurality of persons is displayed by the display provided in a control room or in a ward room. Beneficially, the caregivers in the control room or in the ward room have an overview panel at hand. For example, this would save the caregivers the trouble of actually walking to the door when they want information concerning the sleep state.

These and other characteristics, features and advantages of the present invention will become apparent from the following detailed description, taken in conjunction with the accompanying drawing, which illustrates, by way of example, the principles of the invention. The description is given for the sake of example only, without limiting the scope of the invention. The reference figures quoted below refer to the attached drawing.

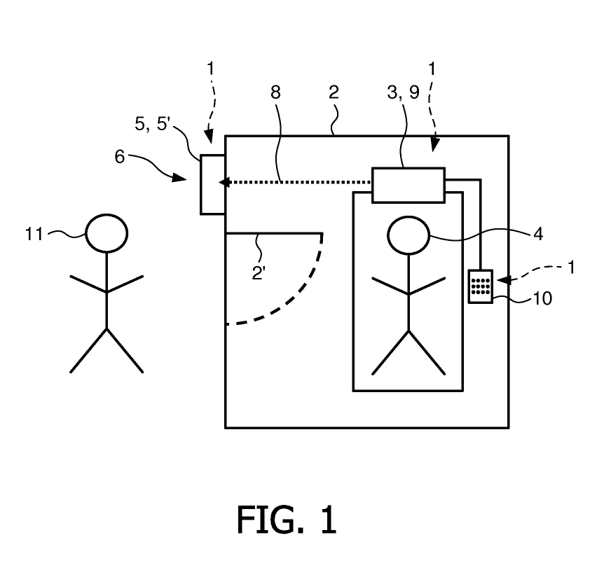

FIG. 1 schematically illustrates an entrance information system, according to the present invention.

The present invention will be described with respect to particular embodiments and with reference to a certain drawing but the invention is not limited thereto but only by the claims. The drawing described is only schematic and is non-limiting. In the drawing, the size of some of the elements may be exaggerated and not drawn on scale for illustrative purposes.

Where an indefinite or definite article is used when referring to a singular noun, e.g. “a”, “an”, “the”, this includes a plural of that noun unless something else is specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the present description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

In FIG. 1 a schematic illustration of an entrance information system 1, according to the present invention is shown. The entrance information system 1 comprises a sleep detector 3 for detecting the sleep stage of a first person 4 in a sleeping room 2. The sleep detector 3 is connected with a control unit 9. The control unit 9 receives and analyzes a sleeping signal from the sleep detector 3 and distinguishes the current sleep stage of the first person 4. Particularly, the control unit 9 distinguished whether the first person 4 is sleeping in REM sleep phase (Rapid Eye Movement), in deep sleep phase or in shallow sleep phase. The detected current sleep stage of the first person 4 is sent to an output unit 5 by a wireless connection 8 via WLAN, Bluetooth, infrared or the like. The output unit 5 is provided outside the sleeping room 2 and comprises a display 5′ adjacent to the door 2′ of the sleeping room 2. The output unit 5 displays an entrance instruction 6 which reflects the sleep state of the first person 4 to signalize it to a second person 11, e.g. a caregiver. Preferably, the display 5′ therefore illuminates in a certain color in accordance to the current sleep phase. Particularly, the entrance instruction 6 reflects the sleep state and combines it with a warning for the second person 11. For example, in case of REM sleep the display unit notifies: “The sleeper is in REM sleep: Safe to go in quietly, but waking not advised.”. In case of deep sleep the display unit notifies: “The sleeper is in deep sleep: Safe to go in quietly, but waking not advised.”. In case of shallow sleep the display unit notifies: “The sleeper is in shallow sleep: This is a good time to wake the sleeper if you need to; otherwise entering not advised.”. And in case of an awake person: “The person is awake. Entering quietly might be appreciated anyway”. Therefore, the second person 11 outside the sleeping room 2 can take the entrance instruction 6 into account in taking the decision whether to enter the sleeping room 2 or not, for example. Also in the case the second person 11 wants to wake up the first person 4, an optimal timing for that is advised by the entrance instruction 6. Further, the entrance information system 1 comprises a user interface 10 for setting up the control unit 9 which allows the first person 4 or the second person 11 to input possible preferences of the sleep like the wakeup time into the control unit 9. The output unit 5 outside the sleeping room 2 reflects the sleep state and further combines it with the preferences of the first person 4. For example, in case that the first person 4 prefers to be left asleep at this hour of day the display notifies: “The sleeper is in shallow sleep and prefers to be left sleeping at this hour: If you need to wake the sleeper this is a good time; otherwise entering not advised.”. And in case that the sleeper prefers to be woken up at this hour of day the display unit notifies: “The sleeper is in shallow sleep and prefers to be woken up at this hour: Now is a good time to do so.”. The user interface 10 is hardwired to the control unit 9.

1. Entrance information system (1) for a sleeping room (2) comprising a sleep detector (3) for detecting a sleep stage of a person (4) in the sleeping room (2), wherein the sleep detector (3) is provided inside the sleeping room (2), and an output unit (5) for issuing an entrance instruction (6) for the sleeping room based on a sleeping signal from the sleep detector (3), wherein the output unit (5) is provided outside the sleeping room (2).

2. Entrance information system (1) according to claim 1, wherein the entrance information system (1) comprises a control unit (9) for receiving the sleeping signal from the sleep detector (3) and for generating the entrance instruction (6).

3. Entrance information system (1) according to claim 2, wherein the entrance information system (1) comprises a user interface (10) for setting up the control unit (9).

4. Entrance information system (1) according to claim 1, wherein the output unit (5) comprises a display (5′) adjacent to a door (2′) of the sleeping room (2) or provided in a control room.

5. Entrance information system (1) according to claim 1, wherein the entrance information system (1) comprises a plurality of sleep detectors (3′) for detecting the sleep stage of a plurality of persons (4) in the sleeping room (2).

6. (canceled)

7. Method according to claim 16, wherein the entrance instruction (6) is allocated by the control unit (9) both in dependence of the sleeping signal and in dependence of an input signal of the user interface (10).

8. Method according to claim 16, wherein the entrance instruction (6) permits the quiet entrance in the sleeping room (2) during REM and deep sleep phases of the person (4).

9. Method according to claim 16, wherein the entrance instruction (6) prohibits the entrance in the sleeping room (2) during shallow sleep phase of the person (4) or permits waking up the person (4) sleeping in the sleeping room (2) during shallow sleep phase of the person (4).

10. Method according to claim 16, wherein an emergency call is activated by the control unit (9) in case of abnormalities in the sleeping signal.

11. Method according to claim 16, wherein the entrance instruction (6) is allocated in dependance of a plurality of sleeping signals (8) from a plurality of sleeping detectors (3, 3′) in the sleeping room (2).

12. Method according to claim 11, wherein the entrance instruction (6) prohibits the entrance in the sleeping room (2), when at least one of the plurality of persons (4) in the sleeping room (2) sleeps in the shallow sleep phase, or permits waking up at least one of the plurality of persons (4) sleeping in the sleeping room (2) who is currently the only one in the shallow sleep phase.

13. Method according to claim 11, wherein the entrance instruction (6) permits the entrance in the sleeping room (2), when every person (4) in the sleeping room sleeps in the REM or in the deep sleep phase.

14. Method according to claim 11, wherein the sleep state of each person (4) of the plurality of persons (4) is displayed by the display (5′) provided in a control room or in a ward room.

15. Method according to claim 16, wherein the method for issuing the entrance instruction (6) for a sleeping room (2) is used in one of a hospital, a hotel, a nursing home, a retirement home, and a nursery.

16. A method for issuing entrance instructions for a sleeping room by an entrance information system, comprising:
detecting, by a sleep detector, a sleep stage of a person in the sleeping room, wherein the sleep detector is provided inside the sleeping room; and
issuing, by an output unit, an entrance instruction for the sleeping room based on a sleeping signal from the sleep detector, wherein the output unit is provided outside the sleeping room.
